(19) United States
(12) Patent Application Publication
(10) Pub. No.: US 2026/0273290 A1
(43) Pub. Date: Sep. 17, 2026

(54) DEFIBRILLATOR

(71) Applicants: Walter Mehnert, Ottobrunn (DE); Thomas Theil, Feldafing (DE); Rainer Reimann, Oberhausen (DE)

(72) Inventors: Walter Mehnert, Ottobrunn (DE); Thomas Theil, Feldafing (DE); Rainer Reimann, Oberhausen (DE)

(21) Appl. No.: 19/566,061

(22) Filed: Mar. 13, 2026

(30) Foreign Application Priority Data
Mar. 15, 2025 (DE) 10 2025 110 017.9
Apr. 7, 2025 (DE) 10 2025 113 660.2
Apr. 9, 2025 (DE) 10 2025 114 064.2
Jun. 26, 2025 (DE) 10 2025 124 883.4
Nov. 21, 2025 (EP) 25217830.6
Feb. 11, 2026 (EP) 26157807.4
Feb. 23, 2026 (EP) 26160218.9
Mar. 2, 2026 (EP) 26161703.9

(51) Int. Cl. A61N 1/378; A61N 1/375

(52) U.S. Cl. CPC A61N 1/3787; A61N 1/37512; A61N 1/37516; A61N 1/3756

(57) ABSTRACT

The application relates to a defibrillator for implantation into a body of a living being and for controlling a cardiac activity of the heart of the living being, including: an electrode section, which is arranged as intended during implantation of the defibrillator such that a current surge/shock (ST) delivered via the electrode section controls the cardiac activity; switching electronics connected to the electrode section, which are configured to control the defibrillator and to trigger delivery of the current pulse (ST); at least one electrical energy storage device which supplies a voltage for long-term power supply, wherein the switching electronics are configured to generate, from the supplied voltage, a surge/shock voltage by means of which the electric current surge/shock for controlling the cardiac activity is delivered, by multiplying and/or reducing the supplied voltage for generating the surge/shock voltage; and an energy receiving section connected to the switching electronics, which is configured to receive energy in a contactless manner and to deliver the energy to the energy storage device for recharging the energy storage device, wherein the energy receiving section includes at least one coil which is configured to receive the energy and to deliver it to the energy storage device via a rectifier when permeated by a magnetic alternating field generated by an external charger.

### FIELD OF THE INVENTION

The invention relates to a rechargeable defibrillator designed for preferably complete implantation into a body of a living being and for controlling a cardiac activity of a heart of the living being. In particular, the invention relates to a defibrillator that is preferably fully implanted into the human body and that controls the cardiac activity of the human heart.

### BACKGROUND OF THE INVENTION

Defibrillators are generally known in the prior art. In addition to external defibrillators, which a physician or rescuer applies externally to the body of a patient in an acute emergency in order to deliver an electrical shock controlling cardiac activity, implantable defibrillators exist.

The latter remain permanently in the body of the patient after implantation, monitor the cardiac activity, and, if necessary, deliver an electrical shock to the heart of the patient.

The implanted defibrillators obtain the energy required for their operation from a battery, the electronics being designed such that the energy content of the battery is sufficient to supply the respective defibrillator with energy until the death of the patient or at least over decades. Should it occur that the energy content of the battery is insufficient, the battery is replaced by performing a surgical intervention. This circumstance alone is already disadvantageous.

In addition, batteries for use in defibrillators have electrical limitations that can only be compensated for by undesirable measures.

The current surge applied by the known defibrillators, for example, occurs at a voltage of +/−400V with not inconsiderable currents. On the one hand, the batteries do not supply such voltages, which is why a transformer is used to increase the voltage supplied by the corresponding battery. If the current surge is to occur simultaneously at high amperage, this would require a current of several amperes on the primary side of the transformer, depending on the winding ratio of the transformer. Known usable batteries do not supply such currents. Withdrawal rates (withdrawable current/charge content; amperes/per ampere hour) of known good batteries are approx. 10 amperes/per ampere hour.

If the transformer requires 1 A at 400 V on its secondary side, this results in 100 A at 4 V on the primary side, for example. In this case, the battery required a charge of 33000 As. Today, this corresponds to a battery volume of 33 ccm. Implantation of such a battery is virtually impossible.

In order to avoid the problems described above, the known implantable defibrillators have storage capacitors which are charged via the secondary side of the transformer at the voltage level mentioned with currents in the mA range until the corresponding storage capacitor can deliver a current surge at the voltage level with the required current strength.

On the one hand, the storage capacitors mentioned have undesirably large dimensions. On the other hand, the loading time is annoying.

Implantation of the known defibrillators in the human heart is ruled out by the restrictions described above, also due to the resulting size.

### BRIEF SUMMARY OF THE INVENTION

Against this background, it is an object of the invention is to provide for an improved defibrillator. At least it is an object of the invention to provide for an alternative defibrillator to the prior art.

This object(s) is/are solved by a defibrillator according to the independent Claim(s). Preferred embodiments are the subject-matters of the dependent Claim(s).

An defibrillator according to the invention for implantation into a body of a living being and for controlling a cardiac activity of the heart of the living being comprises:
an electrode section which is arranged as intended when the defibrillator is implanted in such a way that a current surge/current shock emitted via the electrode section controls cardiac activity;
switching electronics connected to the electrode section, which are configured to control the defibrillator and trigger the delivery of the electric current shock;
at least one electrical energy storage device which supplies a voltage for long-term supply, wherein
the switching electronics are configured to generate, from the supplied voltage, a shock voltage by means of which the electric current shock for controlling the cardiac activity is delivered, by multiplying and/or reducing the supplied voltage for generating the shock voltage; and
an energy receiving section connected to the switching electronics, which is configured to receive energy without contact and to deliver it to the energy storage device for recharging the energy storage device, the energy receiving section having at least one coil which is configured to receive the energy and deliver it to the energy storage device via a rectifier when it is passed through by an alternating magnetic field generated by an external charging device.

The multiplication and/or reduction of the voltage supplied by the at least one electrical energy storage device to generate the shock voltage is preferably carried out with galvanic isolation by a transformer and/or without galvanic isolation by a converter, as will be described below.

The electrode section may have an electrode exposed on the outer surface of the defibrillator housing. Alternatively, the electrode section may have a cable probe that runs by cable from the defibrillator housing to a specific location within the living body, where the electric current shock can be delivered.

The defibrillator according to the invention can be realized in different implantation variants.

According to a preferred first implantation variant, the defibrillator is implanted under the skin next to a muscle or in a muscle. For example, in this implantation variant the defibrillator is implanted under/next to the sternum or under/in the chest muscle.

The electrode section according to this first implantation variant has, for example, at least one cable probe which is guided from the defibrillator housing to a suitable location close to the heart and from there emits the electric current shock. The cable probe therefore does not touch the heart directly (but only indirectly via intervening tissue). Another electrode of the electrode section can be an electrode surface exposed on the defibrillator housing or another cable probe. The defibrillator housing or the additional cable probe is also arranged as intended in such a way that the exposed electrode or the additional cable probe does not touch the heart directly (but only indirectly via intervening tissue). The electrodes are arranged as intended in such a way that the heart is as close as possible to the course of the electric field and experiences a large field effect when the electric current shock is delivered. This first implantation variant is referred to below as the “first indirect implantation variant”.

Alternatively, the electrode section according to this first implantation variant preferably has at least one cable probe, which is guided from the defibrillator housing to an outer heart wall, or at least one, preferably venous, vascular cable probe, which is guided from the defibrillator housing through a vessel into the heart and anchored there. A further electrode of the electrode section can be an electrode surface exposed on the defibrillator housing, a further cable probe guided to the outer wall of the heart, or a further, preferably venous, vascular cable probe. This implantation variant is referred to below as the “second direct implantation variant”.

Preferably, the defibrillator according to a “third preferred cardiac implantation variant” can be a heart capsule which, as intended, is implanted completely in the heart, for example in one of the heart chambers. An respective anchoring is preferably leadless, for example via an anchoring protruding from an outer surface of the heart capsule, for example an anchoring spiral, which is twisted into the heart tissue at the appropriate location as intended. Preferably, the heart capsule has a further linearly guided anchorage.

The electrode section of the third cardiac implantation variant is formed, for example, by electrode surfaces exposed on the outer surface of the heart capsule.

Preferably, the anchoring, for example the anchoring spiral, performs a dual function by also acting as an electrode.

The fact that the energy storage device is a rechargeable energy storage device has several advantages.

For example, according to the third implantation variant, the defibrillator can be built so small that it can be implanted in a heart chamber. In this implantation variant of the defibrillator, the heart capsule has a volume of 0.5 cm³ to 3.0 cm³, in particular of 0.5 cm³ to 1.5 cm³, in particular of 0.8 cm³.

A small volume of the defibrillator can also be achieved in the first and second implantation variants, in that the defibrillator housing, in which, apart from the electrode section—which is understandably arranged outside the housing—, the aforementioned components, which are part of the claim, are accommodated, is between 4 cm³ and 6 cm³, in particular is 5 cm³.

In addition, discharge rates C (1/h) of rechargeable energy storage devices are considerably better than those of batteries, so that there is greater flexibility to select suitable current shock characteristics depending on the setup and implantation variant. For example, despite the low volume of all implantation variants, it is possible to realize electric current shocks with a high charge content in the millisecond range.

The defibrillator according to the invention is preferably configured such that the electric current shock has, for example, a current strength of between 0.2 A and 1 A or between 0.5 A and 2 A, so that the current shock has sufficient charge content in the millisecond range.

Particularly preferably, the charge content in the second direct implantation variant and the third implantation variant as a heart capsule can also be lower, for example from 3 to 4 μAs with 3 ms to 4 ms and 1 mA in the borderline case.

For example, the energy storage device is an electrochemical accumulator or a solid-state accumulator. The energy storage device intended for long-term supply is preferably exclusively a rechargeable energy storage device. This means that no (non-rechargeable) battery is involved in the long-term supply.

The defibrillator can make do with a single accumulator, especially in the third implantation variant, in which the defibrillator is in direct contact with the heart (inside the heart chamber). Due to this spatial proximity, the shock voltage and/or the current intensity/strength of the current shock/surge can be lower, even in the borderline case in the order of magnitude of a normal heart impulse.

Preferably, the switching electronics of the defibrillator are configured to reverse the polarity of the surge/shock voltage when the current surge/shock delivered or the surge/shock voltage of successive current surges/shocks in order to change the direction of the current of the current surge/shock.

The polarity reversal has the advantage of controlling cardiac activity. The polarity reversal is carried out, for example, by a polarity reversal device, which is constructed by a plurality of switches. The switches are, for example, transistors such as IGBTs or MOSFETs.

Preferably, the defibrillator includes control, processing and memory electronics comprising a communication unit adapted to communicate with at least the external charging device for aligning and/or changing the alternating magnetic field to reduce a time required for recharging.

In particular, the communication unit is configured to communicate at least with the external charging device for aligning and/or changing the alternating magnetic field in order to reduce the time required for recharging to the preferably absolute minimum with a defined constant magnetic field of the charging device in each case-by aligning the vector of the alternating magnetic field of the charging device (charging device according to EP 4 035 728 A1) in the direction of the coil axis of the implant.

The communication unit communicates with the charging device via a wireless connection, for example Bluetooth, Low Power Bluetooth or another communication standard. In particular, the communication unit may communicate to the charging device the spatial orientation of the implant or of the energy receiving section, respectively, and/or the charging performance. The charging device can deduce from this whether/how it needs to change the alignment and/or the strength of the alternating magnetic field.

The surge/shock voltage output can be generated without and/or with the multiplication of the voltage supplied by the energy storage devices and/or also with a reduction of said voltage.

Additionally or alternatively, the control, processing and memory electronics having the communication unit may be configured in such a way that the function of the defibrillator and/or the pacemaker function explained below can be enabled in the implanted state of the implant, e.g. via the charging device or radio communication.

This preferred configuration of the control, processing and memory electronics having the communication unit is particularly preferably realized in the third preferred cardiac implantation variant of the defibrillator, i.e. the heart capsule.

This allows the defibrillator (with or without pacemaker function) to be implanted prophylactically, wherein the function of the defibrillator and/or the pacemaker can be enabled at a desired time. As long as no enabling takes place, the function(s) cannot be activated.

However, the switching electronics are preferably set up to multiply and/or reduce the voltage supplied to generate the surge/shock voltage.

There are various possibilities in this respect.

In this respect, the defibrillator preferably comprises: a plurality of electrical energy storage devices that supply the voltage for long-term supply, wherein the switching electronics are configured to connect the energy storage devices in parallel for recharging and, in order to at least partially multiply the voltage, to connect them in series.

For this switching between parallel connection and series connection, the switching electronics comprise a plurality of switches, which in turn may be, for example, transistors such as IGBTs or MOSFETs.

The defibrillator, for example, has four to five energy storage devices. The energy storage devices are preferably respectively electrical accumulators, for example electrochemical accumulators or solid-state accumulators.

In an alternative or additional possibility, the switching electronics of the defibrillator preferably include a transformer which multiplies and/or reduces the voltage supplied for the long-term supply or the partially multiplied voltage, to generate the surge/shock voltage.

A transformation ratio of the transformer may unproblematically have high values, such that the transformer can step up the voltage present on a primary side of the transformer, which is supplied for long-term power supply or is partially multiplied, to high voltages present on a secondary side of the transformer, provided that the charge capacity is sufficient. Even in the case of current surges/shocks with high current intensities/strengths in the ampere range (e.g. 1 A) and the correspondingly even higher current intensities/strengths on the primary side of the transformer, in particular small accumulators can supply the required currents.

As already mentioned, the generation of the surge/shock voltage is done by realizing galvanically isolated circuits by means of a transformer.

The transformer requires an AC voltage on the primary side. The defibrillator is preferably configured such that the voltage supplied for long-term power supply or the partially multiplied voltage is applied, via an inverter of the switching electronics, to a primary-side coil of the transformer, and the multiplied and/or reduced shock voltage is output at a secondary-side coil of the transformer.

The shock voltage output at the secondary-side coil may be rectified by a rectifier of the switching electronics.

In an further alternative or additional possibility, the switching electronics of the defibrillator preferably include a converter which multiplies and/or reduces the voltage supplied for the long-term supply or the partially multiplied voltage, to generate the surge/shock voltage.

Converters without galvanic isolation include switched rechargeable energy storage devices (rechargeable battery cells), step-up and step-down converters, electronic potentiometers and voltage dividers.

The converter is preferably made up of a capacitor, preferably a diode, preferably a switch and a converter coil, wherein the converter coil is connected in a switched manner in a circuit supplied by the energy storage device in such a way that it charges the capacitor up to the surge/shock voltage in order to deliver the current surge/shock. Switching preferably takes place several times and is preferably realized by an electronic switch of the switching electronics, such as a transistor (e.g. IGBT or MOSFET), in particular a field-effect transistor.

Particularly in the third implantation variant, in which the current surge/shock can be delivered at a lower voltage due to the proximity to the heart tissue and the resulting lower contact resistance, the capacitor has a size suitable for the heart capsule. For example, the capacitor is a ceramic capacitor.

The defibrillator is preferably constructed such that the coil of the energy receiving section performs a dual function, in that the coil forms a receiving coil for recharging the energy storage device(s) and forms a primary-side coil of the transformer for multiplying/reducing the voltage supplied for long-term power supply or the partially multiplied voltage.

If the defibrillator has the converter which multiplies and/or reduces the voltage supplied for long-term supply or the partially multiplied voltage, to generate the surge/shock voltage, the dual function consists in particular in the fact that the receiving coil also forms the coil of the converter.

The defibrillator is preferably constructed such that the inverter of the switching electronics is a bidirectional inverter/rectifier which
(i) on the one hand, during recharging, rectifies a charging alternating voltage output by the receiving coil into a charging direct voltage and supplies it to the energy storage device(s), and (ii) on the other hand, during generation of the surge/shock voltage, inverts the voltage supplied for long-term power supply or the partially multiplied voltage, and supplies it to the primary-side coil of the transformer.

As an alternative thereto, the defibrillator is constructed such that the switching electronics, in addition to the inverter which, during generation of the surge/shock voltage, inverts the voltage supplied for long-term power supply or the partially multiplied voltage and supplies it to the primary-side coil of the transformer, comprise a separate rectifier which, during recharging, rectifies a charging alternating voltage output by the receiving coil into a charging direct voltage and supplies it to the energy storage device(s).

By providing a bidirectional inverter/rectifier or separate inverter/rectifier, degrees of freedom arise in the design of the defibrillator with respect to volume and/or costs.

The defibrillator is preferably constructed such that the coil of the energy receiving section comprises a magnetically conductive core, preferably a ferrite.

The magnetically conductive core provides for collecting the magnetic alternating field generated by the charging device and thus for improved charging and preferably also for a small structural size.

Preferably, the core of the coil of the energy receiving section surrounds a transformer core of the transformer.

This configuration and the accommodation of the transformer core within the core of the coil of the energy receiving section are particularly advantageous in that the transformer core, which is likewise a magnetically highly conductive element, has no negative effects on recharging, since there is no risk that the transformer core unintentionally deflects the magnetic alternating field generated for recharging out of or around the coil of the energy receiving section.

The transformer is preferably a core transformer, preferably a toroidal core transformer or a pot core transformer or a hollow cylindrical core transformer.

Alternatively, the magnetically conductive core of the coil of the energy receiving section may simultaneously form the core of the transformer, in particular when the coil of the energy receiving section performs the aforementioned dual function.

The defibrillator is preferably constructed such that the core of the coil forms a housing in which the control, processing and storage electronics and/or the switching electronics, and the energy storage device(s) are accommodated, and onto which the coil of the energy receiving section is wound.

The housing carrying the coil may form a defibrillator housing of the defibrillator or may form an internal housing accommodated in the defibrillator.

The defibrillator is preferably constructed such that the housing forming the core of the coil forms a transformer core of the transformer, and the secondary-side coil of the transformer outputting the surge/shock voltage is wound onto the housing and preferably carries the coil of the energy receiving section.

An insulation, such as for example an insulating paper or insulating film, may be arranged between the coil of the energy receiving section and the secondary-side coil.

As mentioned, the coil of the energy receiving section can take on a dual function and, in addition to the function of the charging coil or receiving coil, respectively, required for recharging the energy storage device(s), can also form the primary-side coil of the transformer or the converter coil. That is, during recharging, the magnetic alternating field induces an alternating voltage in the coil of the energy receiving section, which subsequently delivers a charging current that, after rectification, is supplied to the energy storage device(s). When the defibrillator goes into operation after recharging and delivers the current surge/shock when required, the coil takes over the function of the primary-side coil of the transformer, to which the voltage supplied by the energy storage device(s) for long-term supply is fed after inverting (for multiplication or reduction), or takes over the function of the converter coil.

To this extent, the transformer or the converter and the energy receiving section can make do with two coils.

Alternatively, the coils of the transformer and of the energy receiving section may be implemented as separate coils. That is, the defibrillator comprises three coils: the coil of the energy receiving section, the primary-side coil and the secondary-side coil of the transformer.

The defibrillator is preferably constructed such that, when triggered by the switching electronics, the surge/shock voltage is output via the electrode section and the current surge/shock from the energy storage device(s) or from the secondary-side coil of the transformer is delivered via the electrode section for controlling cardiac activity either directly, without intermediate storage, or directly with only smoothing.

In this respect, without intermediate storage means that no storage capacitor has to be charged with the energy required for the current surge/shock before it is released.

This direct delivery of the current surge/shock from the energy storage device(s) or from the secondary-side coil of the transformer has significant advantages, which are based on the use of the rechargeable energy storage device.

In all implantation variants of the defibrillator, current surges/shocks with high current intensities/strengths can therefore be delivered because rechargeable energy storage devices with high discharge rates exist and are usable. This even applies to the first implantation variant at high voltages.

The energy storage device is preferably an electrical accumulator and, in the first indirect implantation variant, is preferably configured such that it has, for example, an installed charge content between 2000 As and 3000 As, preferably 2500 As. In the first indirect implantation variant, the energy storage device is preferably configured such that it can supply, for example, an electric current of 10 A to 25 A (amperes), or even 10 A to 100 A. If the energy storage device is constructed, for example, from the plurality of energy storage devices, each energy storage device, for example, supplies a voltage of 4 V, has a charge content of 500 As, and is configured such that it can supply 43 mA per As of charge content. Accordingly, the extractable current is on the order of the stated magnitude of 21.5 A, with the total charge content amounting to 2000 As/2500 As in the case of, for example, four/five energy storage devices.

In the first indirect implantation variant, the shock/surge voltage is preferably ±400 V. The multiplication required therefor is preferably carried out by the aforementioned transformer and preferably by connecting, for example, five energy storage devices in series, which supply the voltage resulting from the series connection, of for example 20 V, to the primary-side coil of the transformer. The current surge/shock is preferably 1 A at +/−400V over a duration of 5 ms to 10 ms.

The transformation ratio of the transformer is consequently 20, such that a current of 20 A is required on the primary side of the transformer. Each of the five energy storage devices, for example, can supply this current.

The energy required for controlling cardiac activity and/or for the therapy as a whole over the entire lifetime of the patient is far below the energy content of the energy storage device(s).

It is clear from this that the installed charge content of the energy storage device is necessary for the current intensity/strength, but not for the therapy. Consequently, an exceedingly large charge content is available for other functions, and/or the energy storage device(s), by no means, need to be fully charged in order to ensure control of cardiac activity.

In the second direct implantation variant of the defibrillator or in the third implantation variant as a cardiac capsule, the electrical parameters, surge/shock voltage and current intensity/strength, may be the same as described above. However, this is presumably not necessary, since the current surge/shock is applied directly to the heart due to the contact, the smaller electrode spacing, and the substantially lower contact resistance, and no distances to the heart and unnecessary electrical transition resistances need to be overcome.

For example, the energy storage device(s) may have an overall lower charge content, preferably between 100 As and 250 As, with the supplied voltage being 4 V to 8 V. A microtransformer can step up this voltage to 40 V to 80 V (transformation ratio 10). The current pulse may have a value of 0.1 A, resulting in a current of 1 A in the primary-side coil of the transformer. This current is negligible for the energy storage device(s). The current surge/shock is preferably 0.1 A over a duration of 1 ms to 10 ms.

In addition, the direct delivery of the current surge/shock also allows a variation of a characteristic of the current surge/shock. To this extent, the defibrillator is preferably constructed such that the switching electronics are configured to vary a shape and/or a length of the current surge/shock.

For example, the shape and/or the length or duration of the current surge/shock is varied as a function of individual properties of the living being or as a function of the type of detected faulty cardiac activity.

The defibrillator is preferably configured, in particular in the third implantation variant, to vary the current intensity/strength of the current surge/shock. Preferably, a “gentle/soft” current surge/shock is first delivered, for example directly from the energy storage device(s). This gentle/soft current surge/shock may be delivered, for example, at the stated 0.5 V, 1 V, 4 V to 8 V, with 1 mA for 3 ms to 4 ms.

If this gentle current surge/shock does not control the cardiac activity as desired, the defibrillator increases the shock voltage by a factor (e.g. 10), for example via the transformer, which likewise leads to a corresponding increase of the current. The duration of the current surge/shock can initially remain the same or be varied.

The gentle current surge/shock is delivered in particular in the case of detected cardiac fibrillation in order to stabilize cardiac function. For example, the defibrillator may also detect polarities of the cardiac fibrillation and output a plurality of gentle current surges/shocks with polarities opposite to the polarities of the cardiac fibrillation, which leads to an extinction of the cardiac fibrillation. To this extent, the defibrillator is preferably constructed such that the defibrillator at least partially extinguishes faulty pulses, e.g. of a fibrillating heart, by counter-polarity excitation.

The switching electronics of the defibrillator preferably further comprise: at least one storage capacitor which is charged to the surge/shock voltage and which is dimensioned such that it stores at least the energy for one current surge/shock, wherein the at least one storage capacitor delivers the current pulse via the electrode section when triggered by the switching electronics.

If the explained polarity reversal function is provided, the defibrillator comprises at least two storage capacitors, each of which is responsible for one of the current surges/shocks with opposite polarities. Preferably, with low contact resistance and thus low electrical parameters, the storage capacitors, for example as ceramic capacitors, can be dimensioned so small that they fit into the housing of the third cardiac implantation variant (heart capsule).

The defibrillator according to the invention may, in particular in the second direct implantation variant or in the third cardiac implantation variant as a cardiac capsule, also perform the functions of a pacemaker and vice versa.

The defibrillator according to the invention is preferably configured, for performing the pacemaker function, to set, in particular to lower/reduce, the surge/shock voltage by means of a potentiometer or by reversing an operating mode of the transformer.

The reduced surge/shock voltage serves in particular as normal pacemaker stimulation or as gentle defibrillation. In the embodiments described further below, the current surge/shock corresponding to the reduced surge/shock voltage is referred to as a “gentle current surge/shock”. The gentle current surge/shock is generated at 0.5 V, 1 V, 4 V or 8 V and with a current resulting from the contact resistance (e.g. 1 mA with a pulse length of 1 ms to 4 ms, preferably 3 ms to 4 ms).

In the potentiometer variant, the energy storage device(s) are connected to the electrode section via the potentiometer, for example by passing the transformer or the converter.

In the variant of the reversed operating mode of the transformer, the transformer can be operated bidirectionally. The setting of the “gentle current surge/shock” is carried out in that the voltage supplied by the energy storage device(s), after inversion, is stepped down by the transformer. In this case, the coil of the energy receiving section does not only perform the aforementioned dual function (receiving coil and primary coil), but a triple function in the form of the receiving coil, the primary coil and, after reversal of the operating mode of the transformer, in the form of the secondary coil when reducing the supplied voltage.

If the “gentle current surge/shock” does not lead to normal cardiac function, the operating direction of the transformer is reversed and the delivered current surge/shock is generated with the higher electrical parameters explained above. In other words, the functions of the primary and secondary coils of the transformer are reversed for generating the current surges/shocks.

In addition to the preferred switching electronics, the defibrillator comprises control, processing and memory electronics that control the entire defibrillator and realize the functionalities required for controlling cardiac function. These preferably include controlling of the switching electronics, the transformer, the rectifiers and/or the inverters explained above-where present. Furthermore, the control, processing and memory electronics preferably comprise functionalities for monitoring cardiac function, for example pulse measurement functions, heart rhythm detection functions, biosignal measurement functions and/or ECG functions.

The control, processing and memory electronics also preferably realize the functions of the pacemaker.

The energy storage device or the plurality of energy storage devices serve for long-term power supply of the defibrillator by supplying all components, such as the control, processing and memory electronics, with the necessary electrical energy. The energy content of the energy storage device(s) amounts to such an energy quantity that the energy storage device(s) can supply the defibrillator, preferably including the pacemaker function, with electrical energy over a period of one, one and a half, or two years, wherein recharging preferably takes place after the respective half period (0.5, 0.75 or 1 year).

The defibrillator is particularly preferably a wireless heart capsule.

In this respect, the invention relates to a defibrillator for implantation in a body of a living being and for controlling cardiac activity of the heart of the living being, comprising:
an electrode section which is arranged as intended when the defibrillator is implanted in such a way that a current surge/current shock emitted via the electrode section controls cardiac activity
switching electronics connected to the electrode section, which are configured to control the defibrillator and to trigger delivery of the current surge/shock;
at least one electrical rechargeable energy storage device which supplies a voltage for long-term supply, wherein
the switching electronics are configured to generate, from the supplied voltage, a shock voltage by means of which the electric current surge/shock for controlling the cardiac activity is delivered, by multiplying and/or reducing the supplied voltage for generating the shock voltage; and
an energy receiving section connected to the switching electronics, which is configured to receive energy without contact and to deliver it to the energy storage device for recharging the energy storage device, the energy receiving section having at least one coil which is configured to receive at least the energy and deliver it to the energy storage device via a rectifier when it is passed through by an alternating magnetic field generated by an external charging device;
wherein the defibrillator is a leadless heart capsule which, as intended, is anchored in a heart chamber or an outer wall of the heart.

When using the defibrillator to relax (cardiac fibrillation) or resuscitate the heart or heart muscle, it is particularly desirable that a current path of the emitted current surge/shock runs as far as possible through the heart tissue or heart muscle. To achieve this, the invention also provides a defibrillator system which preferably comprises at least two of the defibrillators described above which interact with each other.

Preferably, both defibrillators forming the defibrillator system interact in that they are configured to communicate with one another, for example in order to coordinate the delivery of the respective current surges/shocks. In particular, the defibrillator system is configured such that the at least two defibrillators deliver their current surges/shocks simultaneously or at different points in time.

Alternatively or additionally, the defibrillators forming the defibrillator system interact in that the at least two defibrillators are implanted as intended in such a manner that they together form a current system in which at least partial currents flow between opposing electrodes of the at least two defibrillators.

This achieves that the current path of at least a partial current flows as far as possible through the cardiac tissue or the cardiac muscle. This partial current arises in that the current surge/shock delivered by one of the defibrillators flows not only to its counter-electrode, but also to a counter-electrode of the other defibrillator and thus necessarily through the cardiac tissue or the cardiac muscle.

The current system can preferably be realized when the defibrillators are located in different cardiac chambers and the defibrillators are preferably arranged spatially such that the respective electrodes and counter-electrodes are arranged linearly one behind the other.

Consequently, the defibrillator system is preferably arranged wherein one of the at least two defibrillators is implanted as intended in an atrial chamber of the heart and another of the at least two defibrillators is implanted in a main chamber/ventricle of the heart. The at least two defibrillators forming the defibrillator system preferably simultaneously form a pacemaker network which assumes the function of a multi-chamber pacemaker. Particularly preferably, the pacemaker network comprises the at least two defibrillators forming the defibrillator system and a third unit which is implanted in a further chamber of the heart. The third unit may likewise be a defibrillator having the features explained above and may be part of the defibrillator system.

The configuration of the defibrillator system with defibrillators located in the cardiac chambers is possible (not least due to the absence of storage capacitors) only with recharable-battery-powered defibrillators. The configuration with two or three defibrillators is physically the most effective, because it covers all possibilities (defibrillator including pacemaker in both chambers) and the electrodes capture the most sensitive and most effective region of the heart or the cardiac muscle. This solution places the least load on the heart because, inter alia, the current density at the poles is reduced compared with conventional solutions.

A further solution consists in arranging only one defibrillator, including a pacemaker, at a preferred location in the heart, for example at the sinus node, wherein the counter-electrode is located either in the respective cardiac chamber or outside the heart.

### BRIEF DESCRIPTION OF THE DRAWINGS

Below, preferred embodiments are explained with reference to the attached figures. The foregoing general explanations with respect to the defibrillator according to the invention apply equally to the embodiments relating to the figures and vice versa.

FIG. 1 shows a perspective elevation view of a defibrillator according to the invention for implantation into a body, wherein the defibrillator is implemented in accordance with the first contactless implantation variant or the first cardiac implantation variant.

FIG. 2 shows a longitudinal section view of a defibrillator according to the invention, wherein the defibrillator is implemented in accordance with the third cardiac implantation variant as a cardiac capsule.

FIGS. 3A, 3B and 3C show circuit diagrams of the defibrillator from FIG. 1 and FIG. 2, wherein the respective circuit diagrams can be implemented in the first indirect implantation variant, the second direct implantation variant or the third cardiac implantation variant.

FIG. 4 shows a circuit diagram of the defibrillator from FIG. 1 or FIG. 2; wherein the circuit diagram is preferably suitable for the second direct or third direct (cardiac) implantation variant of the defibrillator.

FIGS. 5A to 5C each show a circuit diagram of the defibrillator of FIG. 1; wherein the circuit diagram can be realized in the first indirect implantation variant or the second direct implantation variant of the defibrillator.

FIG. 6 shows an exemplary circuit design for multiplying and/or reducing the surge/shock voltage and preferably for realizing a normal heart function.

### DESCRIPTION OF EXAMPLE EMBODIMENTS

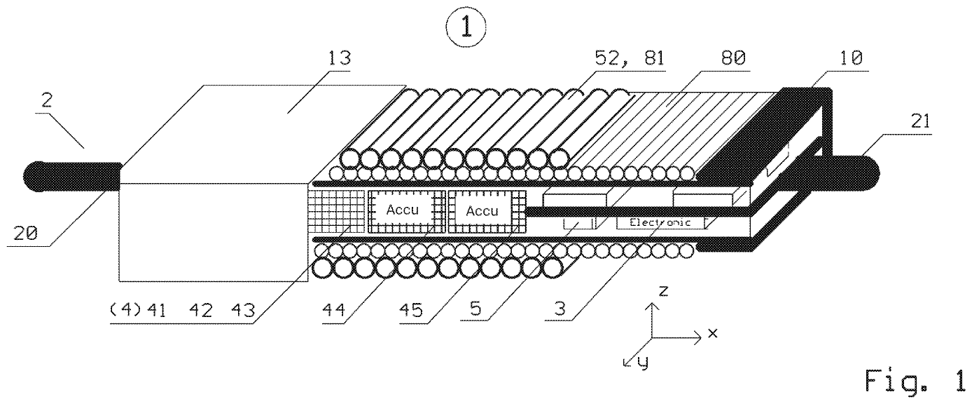

FIG. 1 shows a configuration of a defibrillator 1 according to the invention in a perspective view or a perspective elevation view, respectively, wherein a defibrillator housing 13, which accommodates the illustrated components, is shown.

The defibrillator 1 is preferably fully implanted into a body of a living being, in particular a human being, wherein the defibrillator 1 preferably corresponds to the first indirect implantation variant or the second direct implantation variant.

Preferably all components of the defibrillator 1, except for an electrode section 2, are accommodated in the housing 13 of the defibrillator 1.

The electrode section 2 comprises a connection 20 accessible outside the housing 13 for a (not shown) cable probe. In addition, the electrode section 2 comprises a further electrode 21 which protrudes outwardly from the defibrillator housing 13 and is exposed. It is also possible that a further cable probe is connected as intended to the electrode 21.

Depending on whether the defibrillator 1 is configured in accordance with the first indirect or second indirect implantation variant, at least one cable probe is guided close to the heart or through a vessel into the heart.

The defibrillator housing 13 has a volume of between 4 cm³ and 7 cm³, preferably between 5 cm³ and 6 cm³.

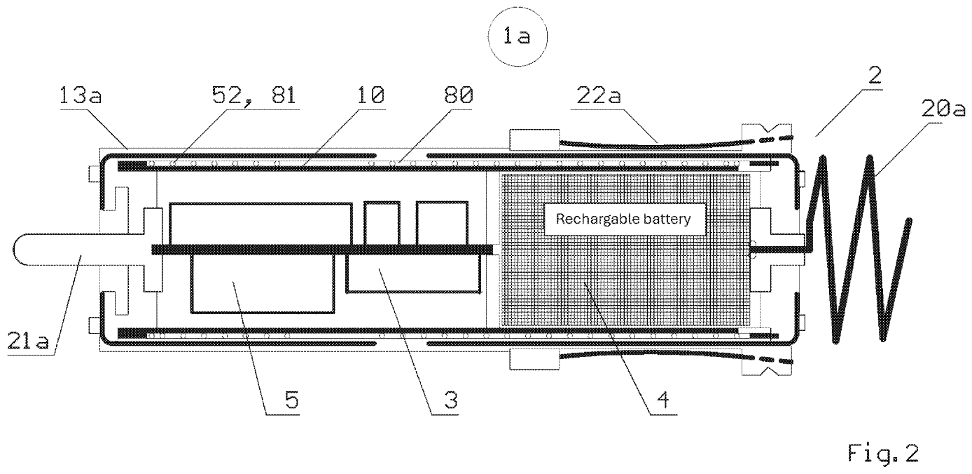

FIG. 2 shows a longitudinal section of a defibrillator 1a according to the third implantation variant.

The defibrillator 1a is in particular a cardiac capsule which is implanted as intended on an outer wall of the heart or within a cardiac chamber.

In the same manner as in FIG. 1, all components are accommodated in a defibrillator housing 13a except for the electrode section 2.

The electrode section 2 comprises an electrode 20a which is designed as an anchoring spiral and is screwed into the cardiac tissue for anchoring the defibrillator 1a. In addition, the electrode section 2 comprises a further electrode 21a which protrudes outwardly from the housing 13a and is exposed.

In particular, this electrode may be implemented as an electrically conductive, prestressed wire which in its normal shape is formed in the shape of a walking stick. This course of the wire serves to reduce the resistance between the two electrodes (the wire and, for example, the spiral 20a). This is to be understood such that the further electrode 20a or the wire in FIG. 2 extends in an arcuate manner from the left-hand side shown there toward the right-hand side of the housing 13a, wherein a distance to the housing 13a slightly increases with increasing proximity to the spiral 20a. An end of the wire facing the spiral 20a may, after anchoring of the defibrillator 1a, contact the cardiac wall tissue. The further electrode 21a may also be formed from a plurality of such wires, for example three or four, which are preferably positioned uniformly around the housing 1a.

The defibrillator 1a according to FIG. 2 is substantially smaller than that of FIG. 1. The defibrillator housing 1a has a volume of 0.5 cm³ or 0.6 cm³ to 3.0 cm³, 0.5 cm³ to 1.5 cm³, preferably from 0.6 cm³ to 1.5 cm³, and particularly preferably from 0.6 cm³ to 0.8 cm³.

In addition to the anchoring spiral functioning as electrode 20a, the defibrillator 1a may have a further, additional anchoring means 22a which comprises linearly displaceable pins. Upon linear displacement of the additional anchoring means 22a, the pins are driven into the cardiac tissue and preferably extend in an arcuate manner. The pins or the additional anchoring means 22a, respectively, may assume the function of the further electrode 21a.

It should be emphasized that the defibrillator 1a of FIG. 2 is entirely free of cable electrodes (leadless), to which negative effects on the tissue are attributed in the case of unfavorable placement.

The defibrillator 1 from FIG. 1 comprises a plurality of electrical energy storage devices 41 to 45. The invention is not limited to this. The defibrillator 1 may also comprise only a single electrical energy storage device 4, in particular in the case in which the defibrillator is implemented in accordance with the second direct implantation variant or the third cardiac implantation variant. FIG. 2 shows the configuration of the defibrillator 1a in the third cardiac implantation variant with only a single energy storage device 4. In principle, the defibrillator 1 in the third cardiac implantation variant may also comprise a plurality of energy storage devices.

The plurality of energy storage devices is not limited to five.

In the following, a plurality of circuit diagrams of the defibrillator are explained.

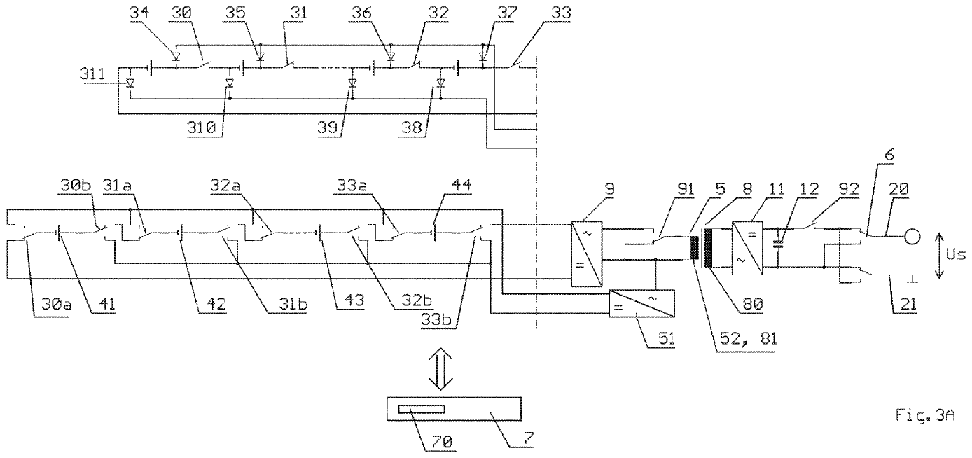

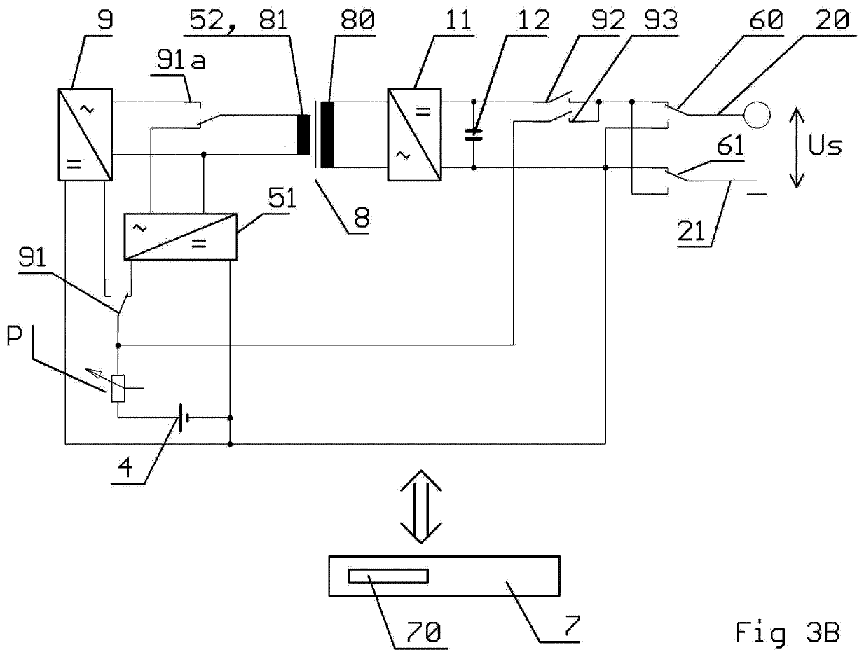

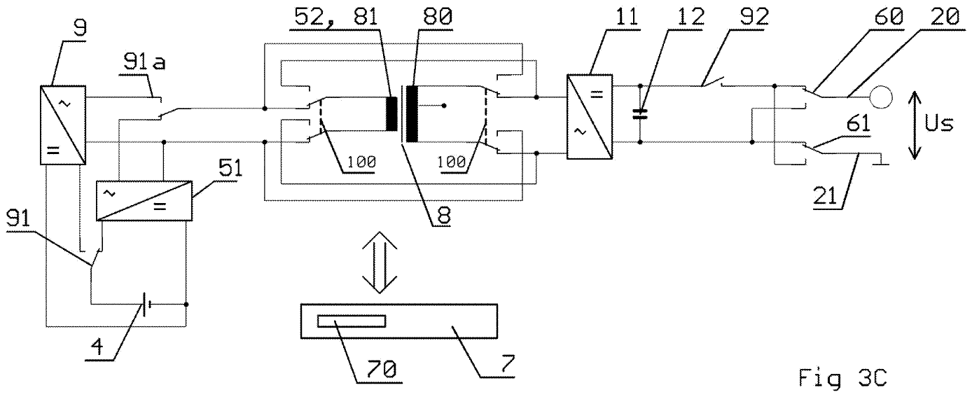

FIGS. 3A, 3B and 3C show circuit diagrams of possible circuit configurations according to which the defibrillator 1 may be implemented in the first indirect implantation variant, the second direct implantation variant and the third cardiac implantation variant.

FIG. 3A shows a circuit configuration of the defibrillator 1, 1a with switching electronics 3 and control, processing and memory electronics 7.

The control, processing and memory electronics 7, hereinafter referred to as CPM electronics 7, control the switching electronics 3, as indicated by the double arrow, and comprise a communication unit 70 for communication with, for example, a charging device or a configuration device.

The defibrillator 1, 1a preferably comprises the plurality of energy storage devices 41 to 45, wherein only four are shown in FIG. 3A for reasons of space. The energy storage devices 41 to 45 are preferably electrical accumulators.

The switching electronics 3 are configured to generate, from the voltage supplied by the energy storage devices 41 to 45, a surge/shock voltage Us with which the current surge/shock for controlling cardiac activity is delivered.

The switching electronics 3 comprise a plurality of switches 30a, 30b, 31a, 31b, 32a, 32b, 33a, 33b via which the CPM electronics 7 can connect the energy storage devices 41 to 45 in parallel or in series.

Furthermore, the switching electronics 3 comprise an inverter 9, a rectifier 51, a transformer 8, a further rectifier 11 and a polarity reversal device 6.

The defibrillator 1, 1a furthermore comprises an energy receiving section 5, which is formed by a (receiving) coil 52 and the rectifier 51.

Preferably, the receiving coil 52 simultaneously forms the primary-side coil 81 of the transformer 8.

From FIG. 1, it can be seen that the primary-side coil 52, 81 is preferably wound around an inner housing 10 present in the defibrillator housing 13, which inner housing 10 is preferably formed from a magnetically conductive material, preferably a ferrite. The inner housing 10 thus forms a core for the coil 52, 81, which collects the magnetic alternating field generated by a charging device. Preferably, the inner housing 10 forms a core of the transformer 8.

A secondary-side coil 80 of the transformer 8 is preferably also wound around the inner housing 10 and is preferably arranged between the primary-side coil 52 and the inner housing 10. To this extent, the secondary-side coil 80 carries the primary-side coil 81.

In FIG. 2 or in the defibrillator 1a according to the third cardiac implantation variant, the receiving coil/primary-side coil 52, 81 is wound, for example together with the secondary-side coil 80, around the inner housing 10, which preferably likewise forms the core of the transformer 8.

In both FIGS. 1 and 2, the receiving coil 52, 81 and the inner housing 10 each perform dual functions and thus contribute significantly to reducing the volume of the defibrillator 1, 1a.

When the defibrillator 1, 1a is being charged, the CPM electronics 7 connect the energy storage devices 41 to 45 in parallel with one another by correspondingly actuating the switches 30a, 30b, 31a, 31b, 32a, 32b, 33a, 33b and switching them from the position shown in FIG. 3A into the respective other position.

The magnetic alternating field generated by the charging device induces a voltage in the receiving coil 52, which generates a charging current. The rectifier 51 rectifies the charging current and supplies it to the energy storage devices 41 to 45 connected in parallel for recharging.

After sufficient charging, for example full charging, the CPM electronics 7 switch into an operating mode and, in this process, connect the energy storage devices 41 to 45 in series by switching the switches 30a, 30b, 31a, 31b, 32a, 32b, 33a, 33b back into the position shown in FIG. 3A, and connect the inverter 9 to the primary-side coil 81 of the transformer 8 by switching the switch 91. Connecting the energy storage devices 41 to 45 in series results in a multiplication of a voltage supplied by the energy storage devices 41 to 45.

In the operating mode, the CPM electronics monitor the heart of the living being, in particular the human being, and decide to deliver a current surge/shock ST to the body via the electrode section 2 when a faulty cardiac activity is detected.

For generating and delivering the current surge/shock ST, the CPM electronics 7 actuate the inverter 9 in order to generate an alternating voltage applied to the primary-side coil 81.

The transformer 8 steps up this voltage into an alternating voltage output at the secondary-side coil 80, which the further rectifier 11, actuated by the CPM electronics 7, rectifies into a surge/shock voltage (direct voltage). The surge/shock voltage and the current surge/shock preferably pass via a smoothing capacitor 12, such that they are preferably additionally smoothed.

The rectified surge/shock voltage is multiplied relative to the voltage supplied by the energy storage devices 41 to 45, wherein connecting the energy storage devices 41 to 45 in series represents a partial multiplication and the transformation by the transformer 8 represents a further multiplication. The transformation or multiplication, respectively, of the supplied or partially multiplied voltage by the transformer 8 into the output and subsequently rectified voltage can generally be seen as a multiplication of the relevant voltage utilizing “galvanic isolation”.

In the preferred first indirect implantation variant, the current surge/shock may have the following electrical parameters.

The surge/shock voltage is, for example, +/−400 V and drives the current surge/shock delivered via the electrode section with a magnitude of 1 A. These currents can be supplied by the electrical energy storage devices 41 to 45 For example, each has a charge capacity of 500 As and a discharge rate of 40-50 mA/As. Consequently, each energy storage device 41 to 45 is capable of supplying a current between 20 and 25 A.

Each energy storage device 41 to 45 additionally supplies a voltage of, for example, 4V, as a result of which a voltage of 20 V is present at the primary-side coil 52, 81 due to the series connection.

With an exemplary transformation ratio of the transformer of 20 and a resulting secondary-side voltage of 400 V, the required primary-side current is 20 A.

An optional switch 92 shown in FIG. 3A is closed during delivery of the current surge/shock.

In contrast, in the second direct implantation variant or the third cardiac implantation variant of FIG. 2, the electrical parameters, direct voltage Us and direct current intensity/strength of the current surge/shock ST, can be substantially lower, since the delivered current surge/shock acts immediately and directly on the heart and the contact resistance present there is lower compared with a contact resistance that the defibrillator 1 in the first contactless implantation variant must overcome by means of the above electrical parameters. It is also assumed that the energy content of the current surge/shock ST can be lower.

For example, the surge/shock voltage Us (direct voltage) is between 50 V and 150 V, in particular between 50 V and 80 V, and the current intensity/strength of the current surge/shock is for example 0.1 A to 1 A. The CPM electronics 7 control the charge content of the current surge/shock ST by controlling the pulse length PL (e.g. 1 ms to 10 ms).

With this in mind, the required charge content of the energy storage devices 41 to 45 can potentially be reduced if the circuit design is used for the second direct or the third cardiac implantation variant. For example, the charge content of the energy storage devices can be reduced to 100 As to 250 As. It is also possible to reduce the number of energy storage devices 41 to 45, for example to provide only a single energy storage device, as shown in FIG. 2. The switches 30a, 30b, 31a, 31b, 32a, 32b, 33a, 33b are correspondingly reduced.

FIG. 3A shows, in its upper part, a modification of the switching electronics 3, which consists in that diodes 34, 35, 36, 37, 38, 39, 310, 311, which may for example be simple PN diodes, are used for multiplying the voltage of the energy storage devices 41-45. As a result, the number of switches 30a, 30b, 31a, 31b, 32a, 32b, 33a, 33b can be reduced by 50%, such that only the illustrated switches 30, 31, 32, 33 remain. The remaining part of the circuit configuration (to the right of the dashed line) is identical to the circuit configuration already explained and is therefore not shown in connection with the modification.

As is apparent from the explanations above, the explained delivery of the current surge/shock is carried out according to FIG. 3A directly from the energy storage devices 41 to 45, merely with preferred transformation and smoothing, without a storage capacitor having to be charged with the energy required for the current surge/shock prior to delivery.

This has substantial advantages, which consist, for example, in that the volume of the defibrillator 1, 1a can be significantly smaller without storage capacitors and that multiple current surges/shocks can be delivered without being limited by time required for intermediate storage.

Furthermore, the charge quantity of the current surge/shock ST can be changed by varying the pulse length PL.

The CPM electronics 7 are preferably configured to temporally vary the shape and/or in particular the duration or pulse length PL, respectively, of the current surge/shock, for example as a function of individual properties of the living being or as a function of the type of detected faulty cardiac activity.

The polarity reversal device 6 enables polarity reversal of successive current surges/shocks. This is carried out, for example, in that the CPM electronics 7 correspondingly actuate two switches 60, 61 of the polarity reversal device. Activating properties are attributed to the polarity reversal of the current pulses.

FIG. 3B shows a further circuit diagram of a circuit configuration according to which the defibrillator 1, 1a may be constructed.

The illustrated circuit configuration is suitable for all implantation variants, in particular however for the second direct implantation variant from FIG. 1 and the third cardiac implantation variant according to FIG. 2.

The circuit configuration shown in FIG. 2 comprises only a single energy storage device 4, which for example has a charge content of 250 As.

Alternatively, the defibrillator 1 may comprise the plurality of energy storage devices 41 to 45 as shown in FIG. 1. If the defibrillator 1 comprises the plurality of energy storage devices 41 to 45, these can in turn be connected in parallel for recharging and connected in series for operation of the defibrillator 1, 1a.

For the sake of simplification, the following description assumes a single energy storage device 4.

When the defibrillator 1, 1a is charged with the circuit configuration according to FIG. 3B, the CPM electronics 7 switch the switch 91 into the position shown in FIG. 3B.

A magnetic alternating field generated by the charging device induces a voltage in the receiving coil 52, which subsequently generates and delivers a charging current.

The rectifier 51 rectifies the charging current and supplies it to the energy storage device 4 for recharging.

After sufficient charging, for example full charging, the CPM electronics 7 switch into an operating mode by switching the switches 91, 91a and thereby connecting the energy storage device 4 to the inverter 9 and connecting the inverter 9 to the primary-side coil 81 of the transformer 8.

In the operating mode, the CPM electronics 7 monitor the heart of the living being, in particular the human being, and decide to deliver the current pulse ST to the body via the electrode section 2 when faulty cardiac activity is detected.

For generating and delivering the current surge/shock ST, the CPM electronics 7 actuate the inverter 9 in order to generate an alternating voltage applied to the primary-side coil 81. The transformer 8 steps up this voltage (multiplication) into an alternating voltage output at the secondary-side coil 80, which the further rectifier 11, actuated by the CPM electronics 7, rectifies and smooths into a high-frequency pulsating direct voltage as the surge/shock voltage. Multiplication by transformer 8 is generally achieved by utilizing the galvanic isolation of the circuits involved. The surge/shock voltage (Us) and the current surge/shock are preferably delivered via the smoothing capacitor 12. The frequency is preferably between 100 kHz and 200 kHz.

When the current surge/shock is delivered via the transformer 8, the switch 92 is closed and a further switch 93 is open.

The energy storage device 4 supplies, for example, 4 V or 8 V.

For example, the surge/shock voltage Us (direct voltage) is between 40 V and 150 V, in particular between 40 V and 80 V, and the current intensity/strength of the current surge/shock is, for example, 0.1 A. The CPM electronics 7 control the charge content of the current surge/shock ST by controlling the pulse length PL.

The defibrillator 1, 1a with the circuit configuration according to FIG. 3B preferably has an additional function in that the current surge/shock ST can be delivered directly from the energy storage device 4 to the heart. This current surge/shock is a “gentle current surge/shock”, for example when stimulating as a “normal” pacemaker. The corresponding gentle current surge/shock is delivered at a low voltage, for example 0.5 V, 1 V, 4 V or 8 V, and with a current resulting from the contact resistance (e.g. 1 mA with a pulse length of 1 ms to 4 ms, preferably 3 ms to 4 ms). Since the contact resistance in/on the heart is low, a current intensity/strength and effect controlling the cardiac activity are established.

In the case of delivering the gentle current surge/shock, the switch 92 remains open or the CPM electronics 7 open the switch 92 beforehand.

Accordingly, the CPM electronics 7 do not operate the path via the inverter 9, the transformer 8 and the rectifier 11.

Instead, for delivering the gentle current surge/shock, the CPM electronics 7 actuate the switch 93 such that the current surge/shock ST with the desired pulse length PL is delivered directly from the energy storage device 4 to the heart. The CPM electronics 7 can use an optional electronic potentiometer P to control, in particular to reduce, the surge/shock voltage Us and preferably the pacemaker voltage.

The function of the polarity reversal device 6 corresponds to that of FIG. 3A, reference being made to the corresponding explanations.

The defibrillator 1, 1a with the circuit configuration according to FIG. 3B enables in particular a very gentle control of cardiac activity.

If the defibrillator 1a or the CPM electronics 7, respectively, for example, detect cardiac fibrillation, a plurality of gentle current surges/shocks can be delivered successively directly from the energy storage device 4 by actuating the switch 93 by the CPM electronics 7 as described.

Preferably, the CPM electronics 7 can output the gentle current surges/shocks with opposite polarities by actuating the polarity reversal device 6.

Particularly preferably, the CPM electronics 7 are configured to detect polarities of electrical biosignals of the heart generated by the cardiac fibrillation and to output the gentle current surges/shocks with polarities respectively extinguishing the cardiac fibrillation.

If the CPM electronics 7 detect that the gentle current surges/shocks delivered via the switch 93 are insufficient to control the cardiac activity in a desired manner, the CPM electronics 7 put into operation the path via the inverter 9, the transformer 8 and the rectifier 11, such that the current surge/shock or current surges/shocks are delivered with the higher voltages and currents specified above.

In the event of cardiac arrest detected by the CPM electronics 7, the CPM electronics 7 immediately put into operation the path via the inverter 9, the transformer 8 and the rectifier 11.

Although not limited to the circuit configuration of FIG. 3B, this configuration is particularly suitable for the defibrillator 1a also to assume the function of a pacemaker. In other words, this circuit configuration allows the realization of a rechargeable pacemaker implantable in the heart with a defibrillator.

The gentle current surge/shock corresponds to a current surge/shock generated by the pacemaker function for stimulating a missing heartbeat. To this extent, the pacemaker function can also be utilized for the defibrillator, in particular in the case of cardiac fibrillation, wherein the path via the inverter 9, the transformer 8 and the rectifier 11 assumes the above-described function of the defibrillator 1a.

FIG. 3C shows a preferred variant of the circuit configuration from FIG. 3B for the correspondingly preferred defibrillator 1, 1a.

Except for the additional function that the gentle current surge/shock can be delivered, the functions of the circuit configuration from FIG. 3C—particularly charging and delivering the current surge/shock ST in the operating mode via the transformer 8 are identical to those of FIG. 3B. In this respect, reference is made to the corresponding explanations.

By contrast, the implementation of the function of delivering the gentle current surge/shock is different. The potentiometer P mentioned above for reducing the surge/shock voltage Us or the pacemaker voltage, and the corresponding contacting with the polarity reversal device 6 are not necessary.

The function of the potentiometer P for adjusting/influencing/reducing the surge/shock voltage Us and preferably the pacemaker voltage is assumed by the transformer 8. For this purpose, the transformer 8 comprises two switching devices 100 and bypass-contacts, which enable bidirectional operation of the transformer 8.

FIG. 3C shows the circuit configuration when recharging of the energy storage device 4. The switches 91a, 91 are switched such that the energy received by the receiving coil 52 is supplied to the energy storage device 4 via the rectifier 51. Reference is made to the corresponding explanations relating to FIG. 3B.

After recharging, the switches 91, 91a switch into the respective other switching position, such that the energy storage device 4 is connected to the primary side or the primary coil 81, respectively, of the transformer 8 via the inverter 9. In this switching position, the defibrillator 1, 1a is in the operating mode, wherein the defibrillator 1, 1a can, as required and controlled by the CPM electronics 7, deliver the current surge/shock ST from the energy storage device 4 to the heart via the path comprising the inverter 9, the transformer 8, the rectifier 11, the preferably provided smoothing capacitor 12, and the preferably provided polarity reversal device 6.

When the defibrillator 1, 1a performs the function of the gentle current surge/shock with the electrical parameters as stated with respect to FIG. 3B, the CPM electronics 7 switch the switching devices 100 respectively into the switching position opposite to that of FIG. 3C, while the switches 91, 91a remain unchanged.

This results in the transformer 8 being operated in reverse, with the coil 80 forming the primary coil and the coil 81 forming the secondary coil. As a result, the transformer 8 steps down (reduces) the alternating voltage output by the inverter 9, such that the gentle current surge/shock ST is delivered from the coil 81, which in this case functions as the secondary coil, to the heart via the rectifier 11, the preferably provided smoothing capacitor 12, and the preferably provided polarity reversal device 6. To this extent, the coil 81, in contrast to the preceding embodiments, assumes not only a dual function as receiving coil 52 and primary coil 81, but a triple function as receiving coil 52, primary coil 81 and, after switching of the switching devices 100, as a secondary coil for voltage reduction.

This “gentle current surge/shock” generated in this manner serves, as already mentioned with respect to FIG. 3B, as stimulation as in a “normal” pacemaker or as gentle defibrillation. The corresponding gentle current surge/shock is delivered at a low voltage, for example 0.5V, 1V, 4 V or 8V, and with a current resulting from the contact resistance (e.g. 1 mA with a pulse length of 1 ms to 4 ms, preferably 3 ms to 4 ms).

If delivery of the gentle current surge/shock ST does not lead to success, the CPM electronics 7 switch the switching devices 100 back again, such that the current surge/shock ST is delivered with the higher electrical parameters explained above. In this respect, reference is made to the corresponding explanations relating to FIG. 3B.

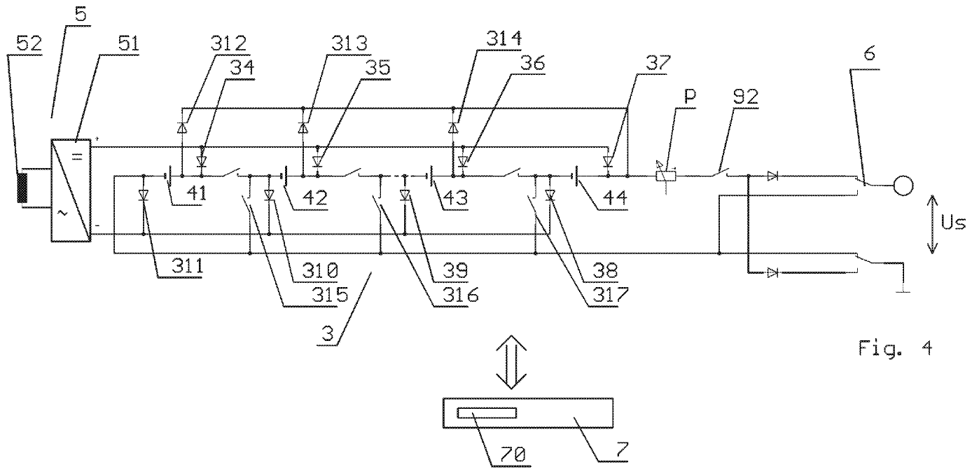

FIG. 4 shows a further circuit configuration according to which the defibrillator 1, 1a may be constructed.

The corresponding circuit configuration is particularly suitable for the defibrillator 1, 1a according to the second direct implantation variant or the third cardiac implantation variant in the form of a cardiac capsule.

The illustrated circuit configuration dispenses with the winding 80.

The receiving coil 52 of the energy receiving section 5 does not perform a dual function but serves exclusively for receiving the energy required for recharging the energy storage devices 41 to 45, which energy is transmitted by the charging device by generating the magnetic alternating field.

The rectifier 51 of the energy receiving section is operated by the CPM electronics 7 for rectifying the alternating voltage output by the receiving coil 52.

The energy storage devices 41 to 45 can be connected in parallel by the switching electronics 3 for recharging and connected in series for operation of the defibrillator.

For this purpose, the switching electronics 3 have the same configuration as the modification shown in the upper part of FIG. 3A. In this respect, reference is made to the corresponding explanations.

In principle, the defibrillator 1 according to the first indirect implantation variant can also be implemented with the illustrated circuit configuration, wherein in this case-at the expense of volume-the plurality of energy storage devices comprises such a number of energy storage devices that, in the series connection, a supplied multiplied voltage of, for example, 400 V results.

Additionally, the circuit configuration comprises the additional diodes 312, 313, 314 and the switches 315, 316, 317, wherein the CPM electronics 7 can, by means of these diodes and switches, exclude individual ones of the energy storage devices 41 to 45 in FIG. 4 from the series connection, starting either from the right-hand side or from the left-hand side, in order thereby to change and reduce, respectively, the surge/shock voltage Us of the delivered current surge/shock. The CPM electronics 7 can use an optional electronic potentiometer P to control, in particular to reduce, the surge/shock voltage Us and preferably the pacemaker voltage. Such an electronic potentiometer P may be present in all circuit configurations shown in FIGS. 3A, 3B and 4.

In general, the defibrillator 1, 1a with a circuit configuration according to FIGS. 3A and 3B can also assume the function of a pacemaker. For delivering the gentle current surge/shock (or a plurality of such gentle current surges/shocks), the corresponding circuit configurations comprise suitable adjustment functions for setting the shock voltage and the current intensity/strength of the gentle current surge/shock.

Although the circuit configurations explained thus far preferably do not comprise storage capacitors and do not require them either, since the currents extractable from the energy storage devices are sufficiently high, the invention is not limited thereto.

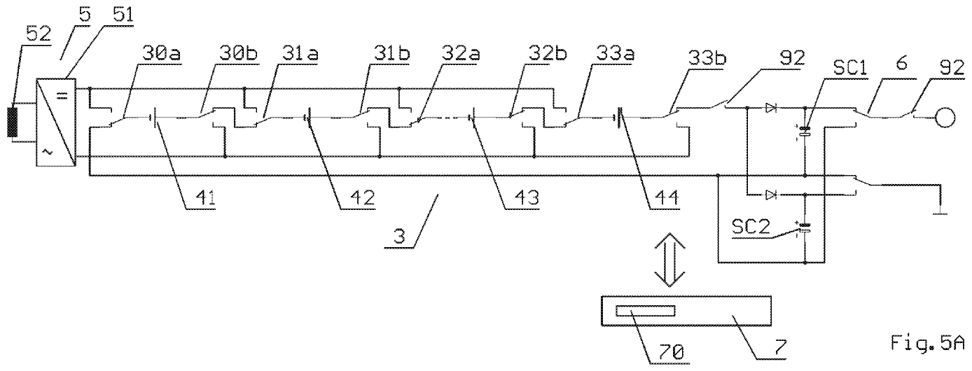

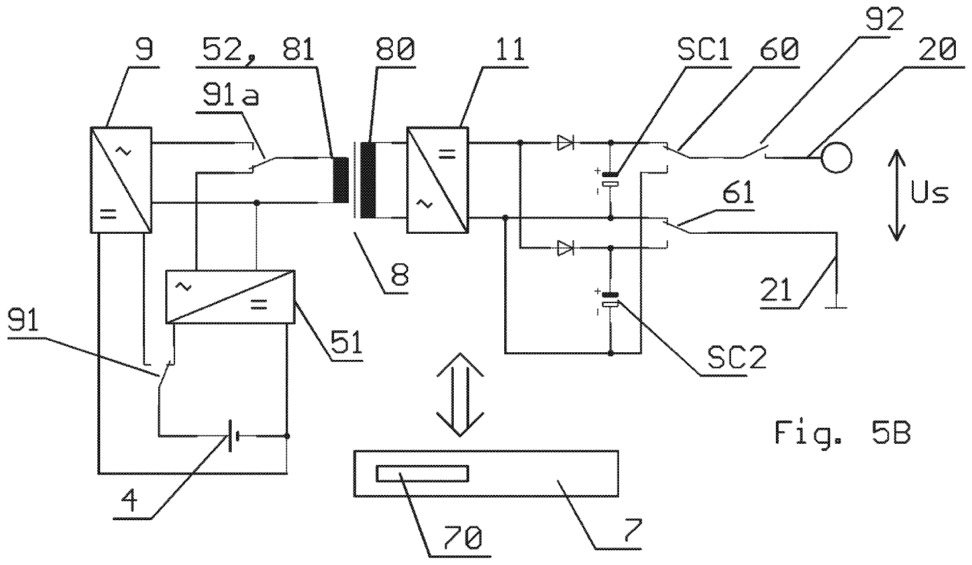

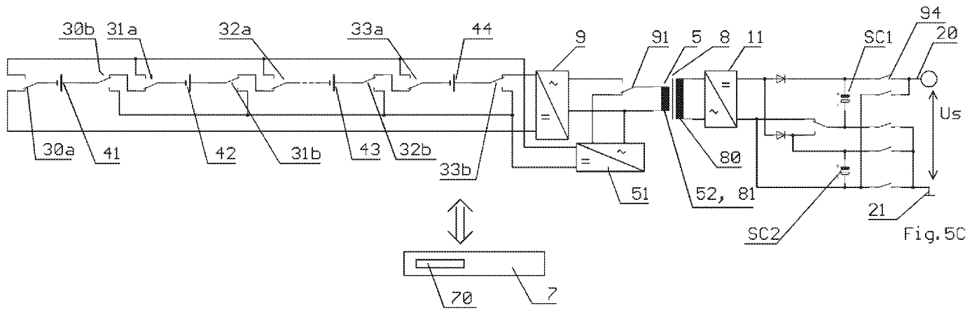

FIGS. 5A to 5C show circuit configurations with storage capacitors. In particular, the defibrillator 1 according to the first indirect implantation variant and the second direct implantation variant may be constructed in accordance with the illustrated circuit configuration.

The circuit configuration according to FIG. 5A dispenses with the winding 80. In particular, the defibrillator 1 according to the second direct implantation variant may have this circuit configuration. If the defibrillator 1 is to be implemented in accordance with the first indirect implantation variant, the number of energy storage devices is increased to such an extent that a multiplied voltage of, for example, 400 V results in the series connection.

The energy receiving section 5 has the same configuration as in FIG. 4, reference being made to the corresponding explanations.

The switching electronics 3, however, have the same configuration as shown in FIG. 3A (lower part), and, in this respect, reference is made to the explanations provided there.

FIG. 5B shows a circuit configuration according to which the defibrillator may be implemented in accordance with the first indirect or the second direct implantation variant. The mode of operation is identical to that of FIG. 3B, with the difference that, on the one hand, the additional function of directly delivering the current surge/shock is not provided, and, on the other hand, storage capacitors are provided.

The circuit configuration according to FIG. 5C is identical to that of FIG. 3A (lower part). In this respect, reference is made to the corresponding explanations. The only difference is that the already optional switch 92 is not present and that storage capacitors SC1/SC2 are provided.

The circuit configurations of FIGS. 5A to 5C each comprise a total of two storage capacitors SC1 and SC2, each of which stores the entire energy for one current surge/shock. The current surges/shocks delivered by the storage capacitors have opposite polarities with respect to one another, wherein the polarity reversal device 6 can switch between the storage capacitors. The delivery of the current surge(s)/shock(s) is controlled by the CPM electronics 7 by means of the illustrated switch combination 94. The circuit configuration of FIG. 5C can additionally generate a doubled surge/shock voltage, however only with a single polarity.

Although not preferred, the storage capacitors SC1 and SC2 could be reduced in size to such an extent that they can also be accommodated in the heart capsule in the third direct implantation variant. This is because the numerical values or sizes, respectively, of the electrical parameters are smaller and therefore the storage capacitors have smaller dimensions. In this case, the storage capacitors SC1 and SC2 are ceramic capacitors, for example.

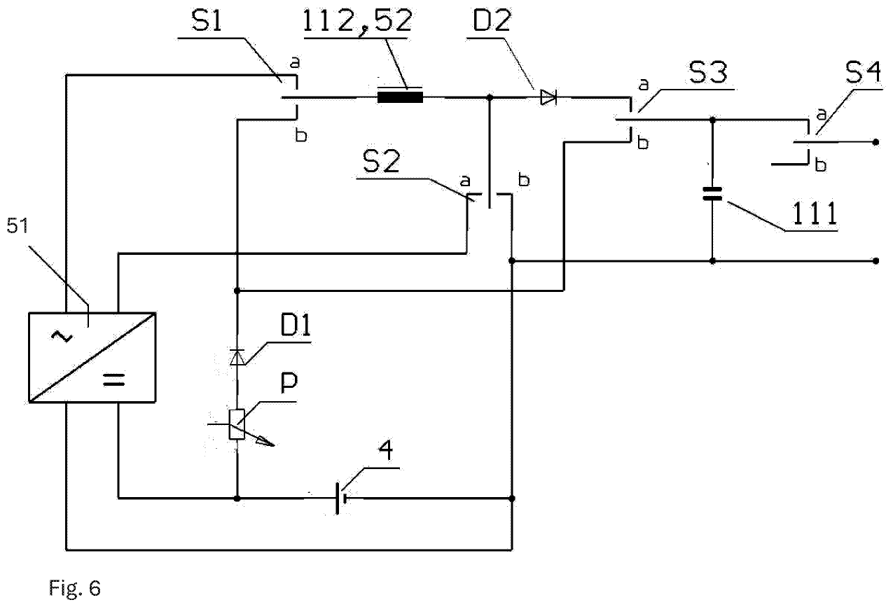

FIG. 6 shows a further circuit configuration that can be used for all implantation variants of the defibrillator 1, 1a, wherein the CPM electronics 7 is not shown.

In contrast to the circuit configurations already described, the voltage supplied by the energy storage device(s) 4 is not multiplied by transformer 8 (galvanic isolation), but by a converter 11. In particular, the switching electronics 3 of the defibrillator 1, 1a include the converter 11, which multiplies the voltage of the energy storage device 4 supplied for the long-term supply (or the already partially multiplied voltage) to generate the surge/shock voltage.

The converter 11 is made up of a capacitor 111, a converter coil 112, a diode D2 and a switch S2, wherein the converter coil 112 is switched in a circuit supplied by the energy storage device 4 in such a way that it charges the capacitor 111 delivering the current surge/shock step by step to the surge/shock voltage by interrupting the switch S2 several times in order.

In addition to the elements of the converter 11 and the energy storage device 4, the circuit configuration has the rectifier 51, the potentiometer P, diode D1 and a plurality of switches S1 to S4 belonging to the switching electronics 3, wherein switch S2 is assigned to the converter 11. The switches S1 to S4 are preferably electronic switches, for example transistors, in particular field-effect transistors (e.g. MOSFETs) or bipolar transistors (e.g. IGBTs).

The rectifier 51, the energy storage device 4 and the potentiometer P have already been described above in connection with the other circuit configurations. Reference is made to the corresponding explanations.

The converter coil 112 acts as the receiving coil 52 of the energy receiving section 5 and thus also performs the dual function explained in this circuit configuration. When the energy storage device 4 is recharged, the switches S1 and S2 are in the respective switch positions a. The alternating voltage generated (induced) by the converter coil 52 and the alternating current driven by it are rectified by the rectifier 51 and fed to the energy storage device 4 for recharging.

To charge the capacitor 111, the switches S1 and S2 first switch to their switching positions b, which causes a current flow from the energy storage device 4 via the potentiometer P, the diode D1 and the converter coil 112. The transformer coil 112 stores energy, by the current flow, in its correspondingly generated field.

The switching electronics 3 then switches the switch S2 to its 0 position and the switch S3 to its switching position a. The interruption of the current flow through the converter coil 112 leads to the reaction known for inductors of seeking to maintain the current flow by the voltage across the converter coil 112 jumping in its polarity and driving the current flow output. An amplitude across the converter coil 112 is dependent on the inductance of the converter coil 112 and the temporal change in the current flow over time, ergo the switching speed of the switch S2.

The current driven by the multiple switching of the converter coil 112 with the corresponding current intensity/strength flows via the diode D2 into the capacitor 111, wherein the capacitor 111 is almost charged to the voltage output by the converter coil 112. The inductance of the converter coil 112 and the switching speed of the switch S2 are dimensioned such that the capacitor 111 is preferably charged to the already explained voltage between 50 V and 150 V, in particular between 50 V and 80 V.

If the defibrillator or the CPM electronics 7, respectively, detects cardiac fibrillation, for example, the current surge/shock can be delivered from the capacitor 11. To do this, the CPM electronics 7 switches the switch S4 to its switching position a, which discharges the capacitor 111. The current strength/intensity of the current surge/shock is 0.1 A to 1 A, for example. The charge content of the current surge/shock ST is controlled by CPM electronics 7 by controlling the pulse length PL (e.g. 1 ms to 10 ms) or the switching duration of switch S4, respectively.

It can be seen from the electrical parameters that the described circuit configuration is particularly suitable for the third cardiac implantation variant. For example, the capacitor 111 is a ceramic capacitor with dimensions that are suitable for the heart capsule.

The defibrillator 1a according to FIG. 6 is also set up to vary the current strength/intensity of the current surge/shock and to deliver the “gentle” current surge/shock, in particular in the third implantation variant. With regard to the electrical parameters when the “gentle” current surge/shock is delivered, reference is made to the corresponding explanations in FIGS. 3A and 3B.

To do this, the CPM electronics set switches S1 and S2 to their 0 positions.

When the “gentle” current surge/shock is to be delivered, the CPM electronics 7 switches switch S3 to its switching position a and switch S4 to its switching position a. The CPM electronics control the pulse length through the switching duration of switch S4. The surge/shock voltage can also be reduced using the potentiometer P.

For all drawings holds: All switches shown in the drawings are preferably not mechanical switches, but electronic switches, in particular transistors, e.g. field-effect transistors (e.g. MOSFETs) or bipolar transistors (e.g. IGBTs).

1. Defibrillator (1) for implantation into a body of a living being and for controlling a cardiac activity of the heart of the living being, comprising:
an electrode section (2), which is arranged as intended during implantation of the defibrillator such that a current surge/shock (ST) delivered via the electrode section (2) controls the cardiac activity;
switching electronics (3) connected to the electrode section (2), which are configured to control the defibrillator and to trigger delivery of the current pulse (ST);
at least one electrical rechargeable energy storage device (4) which supplies a voltage for long-term supply (4), wherein the switching electronics (3) are configured to generate, from the supplied voltage, a surge/shock voltage by means of which the electric current surge/shock for controlling the cardiac activity is delivered, by multiplying and/or reducing the supplied voltage for generating the surge/shock voltage; and
an energy receiving section (5) connected to the switching electronics (3), which is configured to receive energy in a contactless manner and to deliver the energy to the energy storage device (4) for recharging the energy storage device (4), wherein the energy receiving section (5) comprises at least one coil (52) which is configured to receive at least the energy and to deliver it to the energy storage device (4) via a rectifier (51) when it is passed through by an alternating magnetic field generated by an external charging device.

2. The defibrillator according to claim 1, wherein the switching electronics (3) are configured to reverse the polarity of the surge/shock voltage during delivery of the current surge/shock (ST) or to reverse surge/shock voltages of successive current surges/shocks, in order thereby to change a current direction of the current surge/shock (ST).

3. The defibrillator according to claim 1, wherein control, processing and storage electronics (7) comprise a communication unit (70) which is configured to communicate at least with the external charging device with respect to alignment and/or modification of the magnetic alternating field in order to reduce a time required for recharging.

4. The defibrillator according to claim 1, comprising:
a plurality of electrical energy storage devices (41-45) which supply the voltage for long-term power supply, wherein the switching electronics (3) are configured to connect the energy storage devices (41-45) in parallel for recharging and, in order to perform multiplication of the voltage at least partially, to connect them in series.

5. The defibrillator according to claim 1, wherein
the switching electronics comprise a transformer (8) which, for generating the surge/shock voltage, multiplies and/or reduces the voltage supplied for long-term power supply or the partially multiplied voltage.

6. The defibrillator according to claim 5, wherein
the voltage supplied for the long-term supply or the partially multiplied voltage is applied via an inverter (9) of the switching electronics (3) to a primary-side coil (52, 81) of the transformer (8), and, the surge voltage is output at a secondary-side coil (80) of the transformer (8).

7. The defibrillator according to claim 6, wherein
the coil (52, 81) of the energy receiving section (5) performs a dual function, in that the coil forms a receiving coil (52) for recharging the energy storage device(s) and forms a primary-side coil (81) of the transformer (8) for multiplying the voltage supplied for long-term power supply or the partially multiplied voltage.

8. The defibrillator according to claim 7, wherein
the inverter (9) of the switching electronics (3) is a bidirectional inverter/rectifier which
(i) on the one hand, during recharging, rectifies a charging alternating voltage output by the receiving coil (52) into a charging direct voltage and supplies it to the energy storage device(s) (4), and
(ii) on the other hand, when generating the surge/shock voltage, inverts the voltage supplied for long-term power supply or the partially multiplied voltage and supplies it to the primary-side coil (81) of the transformer (8).

9. The defibrillator according to claim 7, wherein
the switching electronics (3), in addition to the inverter (9), which, when generating the surge/shock voltage, inverts the voltage supplied for long-term power supply or the partially multiplied voltage and supplies it to the primary-side coil (81) of the transformer (8), comprise a separate rectifier (51) which, when recharging, rectifies a charging alternating voltage output by the receiving coil (52) into a charging direct voltage and supplies it to the energy storage device(s) (4).

10. The defibrillator according to claim 1, wherein
the coil (52, 81) of the energy receiving section (5) comprises a magnetically conductive core (10), comprising a ferrite.

11. The defibrillator according to claim 10, wherein
the core (10) surrounds a transformer core (82) of the transformer (8).

12. The defibrillator according to claim 11, wherein
the transformer (8) is a core transformer, comprising a toroidal core transformer or a pot core transformer or a hollow cylindrical core transformer.

13. The defibrillator according to claim 10, wherein
the core (10) forms a housing in which the control, processing and memory electronics (7) and/or the switching electronics, and the energy storage device(s) are accommodated, and onto which the coil (52, 81) of the energy receiving section (5) is wound.

14. The defibrillator according to claim 13, wherein
the housing forming the core (10) forms a transformer core (82) of the transformer (8), and
the secondary-side coil (80) of the transformer (8) outputting the surge/shock voltage is wound onto the housing (10) and carries the charging coil.

15. The defibrillator according to claim 1, wherein,
when triggered by the switching electronics (3),
the surge/shock voltage is output via the electrode section (2), and the current surge/shock (ST) is delivered, for controlling the cardiac activity, from the energy storage device(s) (4) or from the secondary-side coil (80) of the transformer via the electrode section (2) directly, merely with smoothing.

16. The defibrillator according to claim 15, wherein
the switching electronics (3) are configured to vary a shape and/or a length (PL) of the current surge/shock (ST).

17. The defibrillator according to claim 1, wherein the switching electronics (3) further comprise:
at least one storage capacitor (13) which is charged to the surge/shock voltage and which is dimensioned such that it stores at least the energy for one current surge/shock (ST), wherein
the at least one storage capacitor (13) delivers the current surge/shock (ST) via the electrode section (2) when triggered by the switching electronics.

18. The defibrillator according to claim 1, wherein the defibrillator additionally assumes a pacemaker function.

19. The defibrillator according to claim 18, wherein the defibrillator is configured, for performing the pacemaker function, to set the surge/shock voltage by means of a potentiometer or by reversing an operating mode of the transformer (8).

20. The defibrillator according to claim 1, wherein the defibrillator at least partially extinguishes faulty pulses, for example of a fibrillating heart, by counter-polarity excitation.

21. The defibrillator system comprising at least two of the defibrillators according to claim 1, wherein both defibrillators are configured to communicate with one another.

22. The defibrillator system according to claim 21, wherein one of the at least two defibrillators is implanted as intended in an atrium of the heart and another of the at least two defibrillators is implanted in a ventricle of the heart.

23. The defibrillator system according to claim 21, wherein the at least two defibrillators deliver their current surges/shocks (ST) simultaneously or at different points in time.

24. The defibrillator system according to claim 21, wherein the at least two defibrillators are implanted as intended such that they together form a current system in which at least partial currents flow between opposing electrodes of the at least two defibrillators.

25. The defibrillator according to claim 1, wherein the defibrillator is a leadless heart capsule which is anchored in a heart chamber or on an outer wall of the heart as intended.

26. The defibrillator according to claim 1, wherein a control, processing and memory electronics (7) has a communication unit (70) which is set up in such a way that the function of the defibrillator or the function of the pacemaker must be enabled.
